(19) United States
(12) Patent Application Publication
(10) Pub. No.: US 2023/0368872 A1
(43) Pub. Date: Nov. 16, 2023

(54) ALERT DECISION SUPPORT SYSTEM BASED ON PATIENT QUALITY OF LIFE SURVEY INFORMATION AND RELATED METHODS AND COMPUTER PROGRAM PRODUCTS

(71) Applicant: SPENCER HEALTH SOLUTIONS, LLC, Durham, NC (US)

(72) Inventors: J. Alan Menius, JR., Cary, NC (US); Benjamin A. Ogorek, Raleigh, NC (US); Paul Cunningham, Hillsborough, NC (US)

(21) Appl. No.: 18/317,482

(22) Filed: May 15, 2023

Related U.S. Application Data
(60) Provisional application No. 63/364,757, filed on May 16, 2022.

(51) Int. Cl. G16H 10/20; G06N 5/022

(52) U.S. Cl. CPC G16H 10/20; G06N 5/022

(57) ABSTRACT

A method includes receiving survey information associated with a patient, the survey information comprising quality of life survey answers for questions associated with a quality of life category; automatically processing the survey information using an Artificial Intelligence (AI) alert decision support system to perform operations comprising: generating an embedding for the survey information; and using a model corresponding to the quality of life category to predict a change in a quality of life score for the patient for the quality of life category based on the embedded survey information; comparing the change in the quality of life score for the patient with a threshold; and generating an alert based on the comparison of the change in the quality of life score for the patient with the threshold.

### RELATED APPLICATIONS

The present application claims priority from and the benefit of U.S. Provisional Patent Application Ser. No. 63/364,757, filed May 16, 2022, the disclosure of which is hereby incorporated herein in its entirety.

### FIELD

The present inventive concepts relate generally to health care systems and services and, more particularly, to decision support systems for use in generating medical alerts.

### BACKGROUND

Patient monitoring systems are being used with increasing frequency to deliver quality health care to patients. Monitoring can be done, for example, by measuring and evaluating different health parameters, such as heart rate, vital signs, blood glucose, and neurological indicators. Home monitoring systems and wearable technology including wireless sensors are commonplace and may be remotely connected to a healthcare facility to a track patient's health and set off alarms in adverse situations. Such digital technology may be beneficial in helping health care providers keep track of patients' vital information in a more timely or even real time manner, which may allow the health care providers to make more frequent and time critical recommendations to improve their patients' health.

### SUMMARY

According to some embodiments of the inventive concept, a method comprises: receiving survey information associated with a patient, the survey information comprising quality of life survey answers for questions associated with a quality of life category; automatically processing the survey information using an Artificial Intelligence (AI) alert decision support system to perform operations comprising: generating an embedding for the survey information; and using a model corresponding to the quality of life category to predict a change in a quality of life score for the patient for the quality of life category based on the embedded survey information; comparing the change in the quality of life score for the patient with a threshold; and generating an alert based on the comparison of the change in the quality of life score for the patient with the threshold.

In other embodiments, the method further comprises: communicating the alert to the patient and/or an entity designated by the patient.

In still other embodiments, the entity designated by the patient is a caregiver for the patient, a health care service provider, or a pharmacist.

In still other embodiments, the quality of life category is a health category, an emotion category, or an activity category.

In still other embodiments, the method further comprises: collecting the survey information in concert with distributing a drug product to a patient via an Internet of Things (IoT) device.

In still other embodiments, collecting the survey information comprises: receiving the answers to the quality of life survey questions from the patient via the IoT device.

In still other embodiments, the method further comprises: receiving clinical information associated with the patient; wherein automatically processing the survey information, comprises: automatically processing the clinical information using the Artificial Intelligence alert decision support system to perform operations comprising: generating an embedding for the clinical information; and using the model corresponding to the quality of life category to predict the change in the quality of life score for the patient for the quality of life category based on the embedded survey information and the embedded clinical information.

In still other embodiments, at least a portion of the clinical information is measured via the IoT device.

In still other embodiments, the AI alert decision support system includes an AI QoL algorithm that is trained by determining similarities between predicted quality of life scores and actual quality of life scores based on embeddings of historical survey information associated with both the patient and a plurality of historical patients; updating the AI QoL algorithm based on loss function results associated with the similarities; and generating the model corresponding to the quality of life category based on the updated AI QoL algorithm.

In still other embodiments, the AI QoL algorithm is configured to perform a dynamic factor analysis.

In still other embodiments, generating the embedding for the survey information comprises: generating the embedding for the survey information using one-hot encoding.

In some embodiments of the inventive concept, a system comprises: a processor; and a memory coupled to the processor and comprising computer readable program code embodied in the memory that is executable by the processor to perform operations comprising: receiving survey information associated with a patient, the survey information comprising quality of life survey answers for questions associated with a quality of life category; automatically processing the survey information using an Artificial Intelligence (AI) alert decision support system to perform operations comprising: generating an embedding for the survey information; and using a model corresponding to the quality of life category to predict a change in a quality of life score for the patient for the quality of life category based on the embedded survey information; comparing the change in the quality of life score for the patient with a threshold; and generating an alert based on the comparison of the change in the quality of life score for the patient with the threshold.

In further embodiments, the operations further comprise: communicating the alert to the patient and/or an entity designated by the patient.

In still further embodiments, the quality of life category is a health category, an emotion category, or an activity category.

In still further embodiments, the operations further comprise: collecting the survey information in concert with distributing a drug product to a patient via an Internet of Things (IoT) device.

In still further embodiments, collecting the survey information comprises: receiving the answers to the quality of life survey questions from the patient via the IoT device.

In some embodiments of the inventive concept, a computer program product comprises: a non-transitory computer readable storage medium comprising computer readable program code embodied in the medium that is executable by a processor to perform operations comprising: receiving survey information associated with a patient, the survey information comprising quality of life survey answers for questions associated with a quality of life category; automatically processing the survey information using an Artificial Intelligence (AI) alert decision support system to perform operations comprising: generating an embedding for the survey information; and using a model corresponding to the quality of life category to predict a change in a quality of life score for the patient for the quality of life category based on the embedded survey information; comparing the change in the quality of life score for the patient with a threshold; and generating an alert based on the comparison of the change in the quality of life score for the patient with the threshold.

In other embodiments, the quality of life category is a health category, an emotion category, or an activity category.

In still other embodiments, the operations further comprise: collecting the survey information in concert with distributing a drug product to a patient via an Internet of Things (IoT) device.

In still other embodiments, collecting the survey information comprises:
receiving the answers to the quality of life survey questions from the patient via the IoT device.

Other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including an intelligent alert Decision Support System (DSS) in accordance with some embodiments of the inventive concept;

FIG. 2 is a block diagram of the intelligent alert DSS in accordance with some embodiments of the inventive concept;

FIG. 3 is a diagram that illustrates embedding of QoL survey answers in accordance with some embodiments of the inventive concept;

FIGS. 4 and 5 are flowcharts that illustrate operations of the intelligent alert DSS in accordance with some embodiments of the inventive concept;

FIG. 6 is a data processing system that may be used to implement an intelligent alert DSS in accordance with some embodiments of the inventive concept; and

FIG. 7 is a block diagram that illustrates a software/hardware architecture for use in an intelligent alert DSS in accordance with some embodiments of the inventive concept.

### DETAILED DESCRIPTION

In the following detailed description, numerous specific details are set forth to provide a thorough understanding of embodiments of the inventive concept. However, it will be understood by those skilled in the art that embodiments of the inventive concept may be practiced without these specific details. In some instances, well-known methods, procedures, components, and circuits have not been described in detail so as not to obscure the inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care products and/or services to a patient.

The term “drug product dispensing system,” as used herein, refers to any type of pharmaceutical or medication dispensing system including, but not limited to, automated systems that dispense vials, bottles, containers, pouches, blistercards, or the like with drug product, semi-automated systems that dispense vials, bottles, containers, pouches, blistercards, strip packages, or the like with drug product, and any combination of automated and semi-automated systems for dispensing a drug product package containing drug product. Drug product dispensing systems also include dispensing systems for pharmaceutical alternatives, such as nutraceuticals and/or bioceuticals.

The terms “pharmaceutical” and “medication,” as used herein, are interchangeable and refer to medicaments prescribed to patients either human or animal. A pharmaceutical or medication may be embodied in a variety of ways including, but not limited to, pill form capsule form, tablet form, and the like.

The term “drug product” refers to any type of medicament that can be dispensed within a vial, bottle, container, pouch, blistercard, or the like by automated and semi-automated drug product dispensing systems including, but not limited to, pills, capsules, tablets, caplets, gel caps, lozenges, and the like. Drug product also refers to pharmaceutical alternatives, such as nutraceuticals and/or bioceuticals.

Embodiments of the inventive concept are described herein in the context of a Decision Support System (DSS) that includes one or more Artificial Intelligence (AI) engines for processing patient Quality of Life (QoL) survey information as well as patient clinical information and generating an alert based on changes in the quality of life score for the patient. The embodiments of the DSS are described with respect to the use of one or more machine learning systems. It will be understood, however, that embodiments of the inventive concept are not limited to machine learning implementations of the DSS and that other types of AI systems may be used including, but not limited to, a multi-layer neural network, a deep learning system, a natural language processing system, and/or computer vision system. Moreover, it will be understood that the multi-layer neural network is a multi-layer artificial neural network comprising artificial neurons or nodes and does not include a biological neural network comprising real biological neurons. The AI engines described herein may be configured to transform a memory of a computer system to include one or more data structures, such as, but not limited to, arrays, extensible arrays, linked lists, binary trees, balanced trees, heaps, stacks, and/or queues. These data structures can be configured or modified through the AI training process to improve the efficiency of a computer system when the computer system operates in an inference mode to make an inference, prediction, classification, suggestion, or the like in response to input information or data provided thereto.

Home monitoring technology has allowed providers to keep track their patients' vial information in a timely manner without being limited to collecting such information only during infrequent office visits. In addition to this clinical information, much can be gleaned about a patient's health based on verbal feedback obtained from a patient. While providers can use telehealth sessions to virtually meet and receive feedback from a patient regarding any health problems or concerns the patient may have, these sessions may be difficult and/or expensive to schedule frequently. Some embodiments of the inventive concept may provide a DSS that includes one or more AI engines for processing patient QoL survey information as well as patient clinical information and generating an alert based on predicted changes in the quality of life score for the patient. Separate AI engines may be devoted to different categories of QoL survey answers, such as a health category, an emotion category, and/or an activity category. The AI engine for a particular QoL survey category may generate a prediction in a change of a QoL score for the patient. This predicted change in the QoL score may be compared to a threshold and, based on this comparison, an alert may be generated that is indicative of a possible need for the patient to take action with respect to the patient's health in a particular QoL category.

The alert may be communicated to one or more people designated by the patient. For example, the alert may be communicated to the patient, a caregiver for the patient, a provider, and/or a pharmacist.

To facilitate collecting the survey information, the survey answers may be collected via an Internet of Things (IoT) device, such as a drug product dispensing device that is used by the patient to dispense prescription medications. For example, a patient may have a drug product dispensing system, which serves as an IoT smart hub in the home for dispensing medications in roll, pouches, or the like to assist a patient in remembering times and dosages for taking medications. Such a drug product dispensing system may be further configured to administer a survey to the user with QoL questions covering one or more of the health, activity, or emotion categories to gain insight on the state of the patient's health in one or more of these QoL areas. In some embodiments, the questions used in the survey may be derived from the questions used in the Flanagan Quality of Life Scale (QOLS).

In some embodiments, the patient survey information for a QoL category may be combined with clinical information for the patient, and the AI engine for the QoL category may generate a prediction in a change of a QoL score for the patient based on both the survey information and the clinical information. The clinical information may provide support that helps to clarify one or more of the answers to the survey questions.

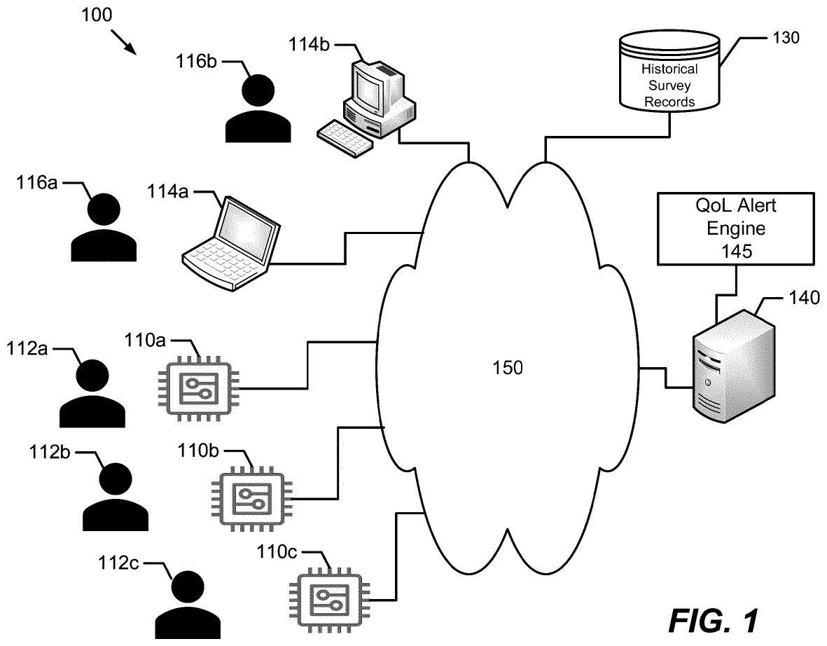

Referring to FIG. 1, a communication network 100 including an intelligent QoL alert DSS, in accordance with some embodiments of the inventive concept, comprises an alert generation server 140, which includes a QoL alert engine module 145. The intelligent alert DSS provided through the alert generation server 140 and the QoL alert engine module 145 may be configured to assist a patient, provider, or other caregiver in making QoL assessments. The alert generation server 140 and the QoL alert engine module 145 may be configured to receive survey information from one or more patients 112a, 112b, and/or 112c via IoT devices 110a, 110b, and/or 110c, respectively. According to some embodiments, these IoT devices 110a, 110b, and/or 110c may each be implemented as an IoT smart hub that is used for dispensing medications for the associated patient 112a, 112b, and/or 112c Each IoT device 110a, 110b, and/or 110c may be further configured to administer a survey to the user with QoL questions covering one or more QoL, categories, such as health, activity, or emotion to gain insight on the state of the patient's health in one or more of these QoL areas. These survey records for various patients 112a, 112b, and/or 112c may be stored in a database 130 where the alert generation server 140 and the QoL alert engine module 145 can process them for training one or more AI models used for assessing patient QoL. The one or more AI models may correspond to the various QoL categories, e.g., health, activity, emotion, etc. In some embodiments, the questions used in the survey may be derived from the questions used in the Flanagan Quality of Life Scale (QOLS). By using the IoT devices 110a, 110b, and/or 110c, which may be embodied as drug product dispensing hubs, to administer the surveys to the respective patients 112a, 112b, and/or 112c, the likelihood that the patient may complete the survey questions may be increased as the patient is required to interact periodically with the IoT devices 110a, 110b, and/or 110c to receive their medications.

In some embodiments, the IoT devices 110a, 110b, and/or 110c may be further configured to obtain clinical information from the patients 112a, 112b, and/or 112c, which may also be stored in the database 130. The alert generation server 140 and the QoL alert engine module 145 may, in some embodiments, use both the patient survey information as well as the patient clinical information for training one or more AI models used for assessing patient QoL.

The alert generation server 140 and the QoL alert engine module 145 may be configured to generate a prediction in a change of a QoL score for a patient based on the survey information and, in some embodiments, the clinical information. An alert may be generated based on a comparison of the predicted change in the QoL score for the patient, i.e., a predicted trend in the QoL score, with a threshold. The alert may be indicative of a patient's declining well-being in a particular QoL category, which may benefit from attention or intervention by one or more entities. Accordingly, the alert may be communicated to the patient 112a, 112b, and/or 112c and/or other entities, such as a health care service provider 116a by way of device 114a, a caregiver for the patient 116b by way of device 114b or other entities, such as pharmacists, psychiatrists, family members, and the like.

A network 150 couples the alert generation server 140, the IoT devices 110a, 110b, and/or 110c, the alert recipient devices 114a and/or 114b, and the database 130 together. The network 150 may be a global network, such as the Internet or other publicly accessible network. Various elements of the network 150 may be interconnected by a wide area network, a local area network, an Intranet, and/or other private network, which may not be accessible by the general public. Thus, the communication network 150 may represent a combination of public and private networks or a virtual private network (VPN). The network 150 may be a wireless network, a wireline network, or may be a combination of both wireless and wireline networks.

The QoL alert generation service provided through the alert generation server 140 and the QoL alert engine module 145 to automatically generate one or more alerts based on an assessment of a patients QoL using patient survey information may, in some embodiments, be embodied as a cloud service. For example, patients may integrate their IoT devices 110a, 110b, and/or 110c or other device used to provide survey and/or clinical information the QoL alert generation service and access the service as a Web service. In some embodiments, the QoL alert generation service may be implemented as a Representational State Transfer Web Service (RESTful Web service).

Although FIG. 1 illustrates an example communication network including an intelligent QoL alert DSS for assisting patients, providers, and/or other caregivers in making QoL assessments, it will be understood that embodiments of the inventive subject matter are not limited to such configurations, but are intended to encompass any configuration capable of carrying out the operations described herein.

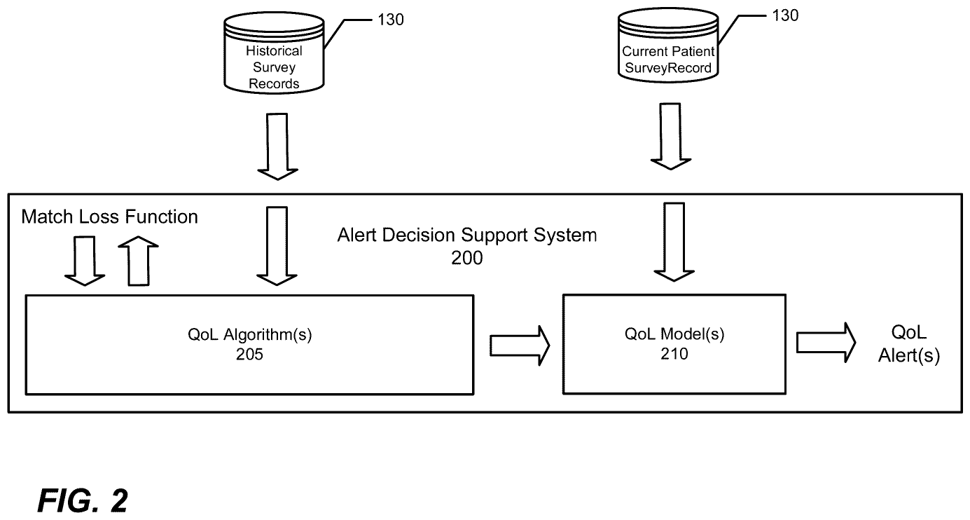

FIG. 2 is a block diagram of an intelligent alert DSS 200 in accordance with some embodiments of the inventive concept. The intelligent alert DSS 200 of FIG. 2 may be used to implement embodiments of the alert generation server 140 and QoL alert engine module 145 of FIG. 1. Referring now to FIG. 2, the intelligent alert DSS 200 includes AI QoL engines or algorithms 205 that may be trained based on historical patient survey records 130 and, in some embodiments, patient clinical information, and are used to generate QoL models 210, which operate in inference mode to generate one or more predicted changes in QoL scores, e.g., trends in QoL scores, based on current patient survey information 130. The AI QoL engines or algorithms 205 and the QoL models 210 may correspond to the different QoL categories being assessed. For example, the AI QoL engines or algorithms may include an engine or algorithm for the health QoL category, an engine or algorithm for the emotion QoL category, and/or an engine or algorithm for the activity QoL category. Like the QoL models 210 may include a QoL model for the health QoL category, a QoL model for the emotion QoL category, and/or a QoL model for the activity QoL category.

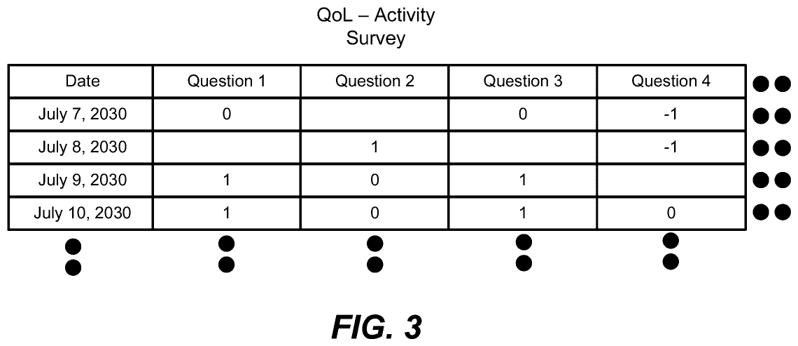

During training mode, the historical patient survey information from the database 130 may be embedded to generate low-dimensional vectors from the high-dimensional survey answer information. For example, FIG. 3 illustrates an embedding for survey answers obtained for a QoL activity category survey. As shown in FIG. 3, an encoding called one-hot encoding is used to generated the embedding by assigning 1 to a positive answer, 0 to a neutral answer, and −1 to a negative answer. Not all questions were answered on each day. By reducing the dimensionality of the survey answers and, in some embodiments, patient clinical information, the processing efficiency in training the QoL engines or algorithms 205 and operating the QoL models 210 in inference mode can be significantly improved. The QoL engine or algorithm for each of the QoL categories may then be trained using the embedded historical survey information by determining similarities between predicted QoL scores and actual QoL scores. In some embodiments, each of the QoL engines or algorithms 205 may be implemented using a dynamic factor analysis. Dynamic factor analysis is a technique used to detect common patterns in a set of time series and relationships between these series and explanatory variables. The various coefficients of the variables used in the dynamic factor analysis may be updated by using a match or loss function to reduce or minimize the similarity differences between the vectors associated with the predicted quality of life scores and trends and the actual quality of life scores and trends from the historical survey information. A match or loss function may be described generally as a function that computes the distance between the current output of an operation and the expected output. In accordance with various embodiments of the inventive concept, the match loss function may comprise a regression loss function or a classification loss function. Broadly described, classification involves prediction of an output from a set of finite categorical values. Regression involves prediction of a continuous value from other information.

The QoL engines or algorithms 205 may be configured to generate the QoL models 210 for the one or more QoL categories, which are each configured to operate at inference time on new patient survey information from the database 130 to generate an alert based on predicted changes in the QoL score for the patient. Similar to the training operations described above, the new patient survey information and, in some embodiments, patient clinical information, may be embedded using, for example, one-hot encoding, which is provided as input to the applicable one of the QoL models 210, i.e., the QoL model 210 that corresponds to the QoL survey information category. The QoL model 210, which has been trained on historical patient survey information including, in some embodiments, the current patient's historical survey information, may be configured to generate a predicted change in the QoL score for the patient and may then compare this predicted change in the QoL score to a threshold to determine whether to generate an alert indicating that the patient's QoL may be declining in this category to such a extent that additional attention may be warranted. The threshold may be adjusted on a patient-by-patient basis to account for differing patient risk levels.

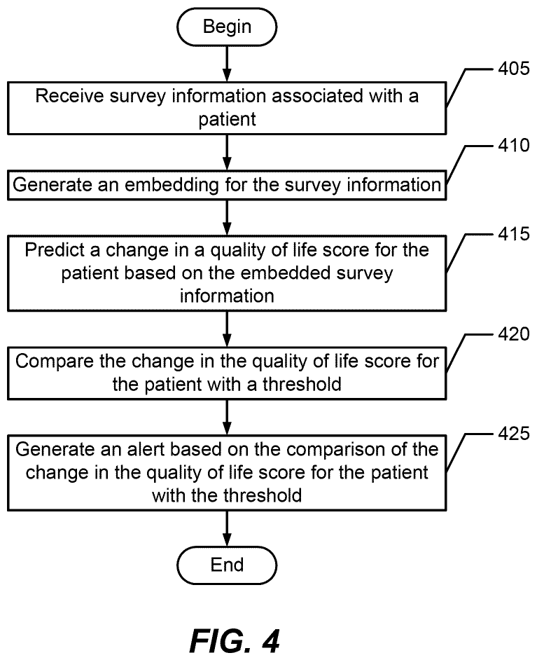

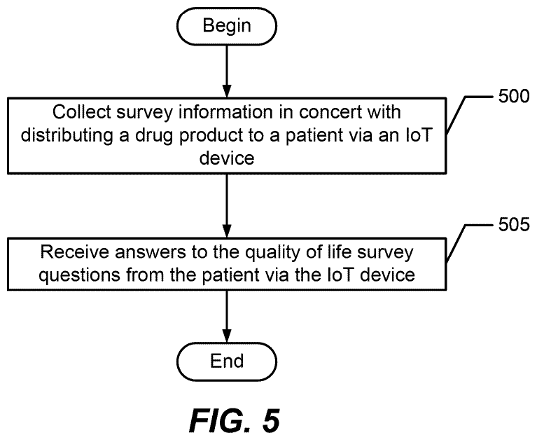

FIGS. 4 and 5 are flowcharts that illustrate operations of the intelligent QoL alert DSS in accordance with some embodiments of the inventive concept. Referring now to FIG. 4, operations begin at block 405 where survey information associated with a patient is received. Referring to FIG. 5, in some embodiments, the survey information may be collected in concert with distributing a drug product to the patient via an IoT device at block 500. The answers to the survey may then be received by the intelligent QoL alert DSS at block 505 for further processing. Returning to FIG. 5, an embedding is generated for the survey information at block 410 using, for example, one-hot encoding. An AI model that corresponds to a QoL category of the survey information may then be used predict a change in a quality of life score for the patient for the quality of life category based on the embedded survey information at block 415. The AI model may be trained based on an AI QoL algorithm that is configured to perform a dynamic factor analysis. The change in the quality of life score for the patient may be compared with a threshold at block 420 and an alert may be generated based on the comparison at block 425. The alert may be communicated to one or more entities, such as the patient, a health care service provider, a care giver, a pharmacist, a family member, etc.

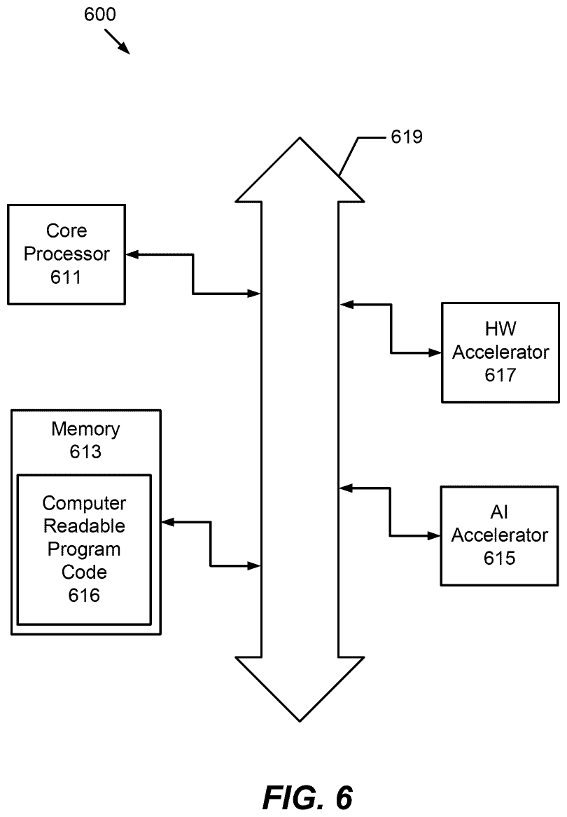

FIG. 6 is a block diagram of a data processing system 600 that may be used to implement the alert generation server 140 of FIG. 1 and the intelligent alert DSS 200 of FIG. 2 in accordance with some embodiments of the inventive concept. As shown in FIG. 6, the data processing system may include at least one core 611, a memory 613, an artificial intelligence (AI) accelerator 615, and a hardware (HW) accelerator 617. The at least one core 611, the memory 613, the AI accelerator 615, and the HW accelerator 617 may communicate with each other through a bus 619.

The at least one core 611 may be configured to execute computer program instructions. For example, the at least one core 611 may execute an operating system and/or applications represented by the computer readable program code 616 stored in the memory 613. In some embodiments, the at least one core 611 may be configured to instruct the AI accelerator 615 and/or the HW accelerator 617 to perform operations by executing the instructions and obtain results of the operations from the AI accelerator 615 and/or the HW accelerator 617. In some embodiments, the at least one core 611 may be an ASIP customized for specific purposes and support a dedicated instruction set.

The memory 613 may have an arbitrary structure configured to store data. For example, the memory 613 may include a volatile memory device, such as dynamic random-access memory (DRAM) and static RAM (SRAM), or include a non-volatile memory device, such as flash memory and resistive RAM (RRAM). The at least one core 611, the AI accelerator 615, and the HW accelerator 617 may store data in the memory 613 or read data from the memory 613 through the bus 619.

The AI accelerator 615 may refer to hardware designed for AI applications. In some embodiments, the AI accelerator 615 may include a machine learning engine configured to facilitate operations associated with an intelligent QoL alert DSS including the various AI engines and AI models described above. The AI accelerator 615 may generate output data by processing input data provided from the at least one core 611 and/or the HW accelerator 617 and provide the output data to the at least one core 611 and/or the HW accelerator 617. In some embodiments, the AI accelerator 615 may be programmable and be programmed by the at least one core 611 and/or the HW accelerator 617. The HW accelerator 617 may include hardware designed to perform specific operations at high speed. The HW accelerator 617 may be programmable and be programmed by the at least one core 611.

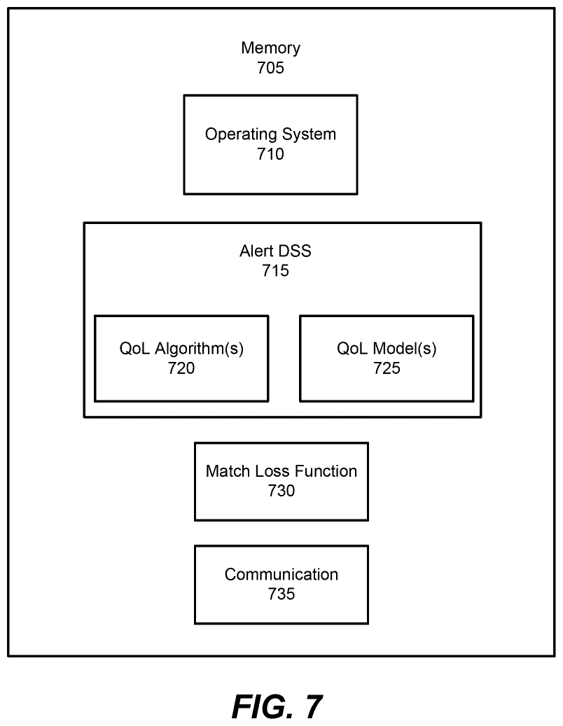

FIG. 7 illustrates a memory 705 that may be used in embodiments of data processing systems, such as the alert generation server 140 of FIG. 1, the intelligent QoL alert DSS of FIG. 2, and the data processing system 600 of FIG. 6, respectively, to facilitate operation of a DSS for assisting patients, providers, and/or other caregivers in making QoL assessments. The memory 705 is representative of the one or more memory devices containing the software and data used for facilitating operations of the alert generation server 140 and the QoL alert engine module 145 as described herein. The memory 705 may include, but is not limited to, the following types of devices: cache, ROM, PROM, EPROM, EEPROM, flash, SRAM, and DRAM. As shown in FIG. 7, the memory 705 may contain four or more categories of software and/or data: an operating system 710, an alert DSS module 715, a match loss function module 730, and a communication module 735. In particular, the operating system 710 may manage the data processing system's software and/or hardware resources and may coordinate execution of programs by the processor.

The alert DSS module 715 may include one or more QoL algorithm modules 720 and one or more QoL model modules 725. The alert DSS module 715 may be configured to perform one or more of the operations described above with respect to the intelligent alert DSS 200 of FIG. 2 and the flowcharts of FIGS. 4 and 5. The QoL algorithm modules 720 may be configured to perform one or more of the operations described above with respect to the QoL engines or algorithm 205 of FIG. 2 and the flowcharts of FIGS. 4 and 5. The QoL model modules 725 may be configured to perform one or more of the operations described above with respect to the QoL model 210 of FIG. 2 and the flowcharts of FIGS. 4 and 5.

The match loss function module 730 may be configured to provide the loss function logic described above with respect to the intelligent alert DSS 200 of FIG. 2. The communication module 735 may be configured to facilitate communication between the alert generation server 140 of FIG. 1 and/or the intelligent alert DSS 200 of FIG. 2 and entities, such as patients, health care providers, patient care givers, pharmacists, patient family members, etc.

Although FIGS. 6 and 7 illustrate hardware/software architectures that may be used in data processing systems, such as the alert generation server 140 of FIG. 1, the intelligent alert DSS 200, and the data processing system 600 of FIG. 6, respectively, in accordance with some embodiments of the inventive concept, it will be understood that the present invention is not limited to such a configuration but is intended to encompass any configuration capable of carrying out operations described herein.

Computer program code for carrying out operations of data processing systems discussed above with respect to FIGS. 1-7 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the alert generation server 140 of FIG. 1, the intelligent alert DSS 200 of FIG. 2, and the data processing system 600 of FIG. 6 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.”

The data processing apparatus described herein with respect to FIGS. 1-7 may be used to facilitate operation of an intelligent QoL alert DSS based on patient QoL survey information according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 705 when coupled to a processor includes computer readable program code that, when executed by the processor, causes the processor to perform operations including one or more of the operations described herein with respect to FIGS. 1-7.

Some embodiments of the inventive concept may provide an intelligent alert generation DSS including one or more AI engines that can be used to process survey information obtained from a patient to predict changes in the patient's QoL score in one or more QoL categories. When a patient's QoL score is trending down by more than a defined threshold, then an alert may be generated to notify the patient and one or more other entities designated by the patient so that attention can be given to any issues that may be negatively impacting the patient's quality of life. In some example embodiments, the survey may be administered by an IoT drug product dispensing hub that the patient uses to control the timing and/or dosage of the patient's medications. This may increase the likelihood that the survey questions are answered on a regular basis, which may improve the assessment of trends in the patient's quality of life in the various categories QoL categories. By assessing a patient's quality of life, a provider may provide improved care to the patient by detecting and addressing areas of the patient's health that may not be easily evaluated based on clinical information alone.

### Further Definitions and Embodiments

In the above-description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

The description of the present inventive concept has been presented for purposes of illustration and description, but is not intended to be exhaustive or limited to the inventive concept in the form disclosed. Many modifications and variations will be apparent to those of ordinary skill in the art without departing from the scope and spirit of the inventive concept. The aspects of the inventive concept herein were chosen and described to best explain the principles of the inventive concept and the practical application, and to enable others of ordinary skill in the art to understand the inventive concept with various modifications as are suited to the particular use contemplated.

What is claimed is:

1. A method, comprising:
receiving survey information associated with a patient, the survey information comprising quality of life survey answers for questions associated with a quality of life category;
automatically processing the survey information using an Artificial Intelligence (AI) alert decision support system to perform operations comprising:
generating an embedding for the survey information; and
using a model corresponding to the quality of life category to predict a change in a quality of life score for the patient for the quality of life category based on the embedded survey information;
comparing the change in the quality of life score for the patient with a threshold; and
generating an alert based on the comparison of the change in the quality of life score for the patient with the threshold.

2. The method of claim 1, further comprising:
communicating the alert to the patient and/or an entity designated by the patient.

3. The method of claim 2, wherein the entity designated by the patient is a caregiver for the patient, a health care service provider, or a pharmacist.

4. The method of claim 1, wherein the quality of life category is a health category, an emotion category, or an activity category.

5. The method of claim 1, further comprising:
collecting the survey information in concert with distributing a drug product to a patient via an Internet of Things (IoT) device.

6. The method of claim 5, wherein collecting the survey information comprises:
receiving the answers to the quality of life survey questions from the patient via the IoT device.

7. The method of claim 5, further comprising:
receiving clinical information associated with the patient;
wherein automatically processing the survey information, comprises:
automatically processing the clinical information using the Artificial Intelligence alert decision support system to perform operations comprising:
generating an embedding for the clinical information; and
using the model corresponding to the quality of life category to predict the change in the quality of life score for the patient for the quality of life category based on the embedded survey information and the embedded clinical information.

8. The method of claim 7, wherein at least a portion of the clinical information is measured via the IoT device.

9. The method of claim 1, wherein the AI alert decision support system includes an AI QoL algorithm that is trained by determining similarities between predicted quality of life scores and actual quality of life scores based on embeddings of historical survey information associated with both the patient and a plurality of historical patients;
updating the AI QoL algorithm based on loss function results associated with the similarities; and
generating the model corresponding to the quality of life category based on the updated AI QoL algorithm.

10. The method of claim 9, wherein the AI QoL algorithm is configured to perform a dynamic factor analysis.

11. The method of claim 1, wherein generating the embedding for the survey information comprises:
generating the embedding for the survey information using one-hot encoding.

12. A system, comprising:
a processor; and
a memory coupled to the processor and comprising computer readable program code embodied in the memory that is executable by the processor to perform operations comprising:
receiving survey information associated with a patient, the survey information comprising quality of life survey answers for questions associated with a quality of life category;
automatically processing the survey information using an Artificial Intelligence (AI) alert decision support system to perform operations comprising:
generating an embedding for the survey information; and
using a model corresponding to the quality of life category to predict a change in a quality of life score for the patient for the quality of life category based on the embedded survey information;
comparing the change in the quality of life score for the patient with a threshold; and
generating an alert based on the comparison of the change in the quality of life score for the patient with the threshold.

13. The system of claim 12, wherein the operations further comprise:
communicating the alert to the patient and/or an entity designated by the patient.

14. The system of claim 12, wherein the quality of life category is a health category, an emotion category, or an activity category.

15. The system of claim 12, wherein the operations further comprise:
collecting the survey information in concert with distributing a drug product to a patient via an Internet of Things (IoT) device.

16. The system of claim 15, wherein collecting the survey information comprises:
receiving the answers to the quality of life survey questions from the patient via the IoT device.

17. A computer program product, comprising:
a non-transitory computer readable storage medium comprising computer readable program code embodied in the medium that is executable by a processor to perform operations comprising:
receiving survey information associated with a patient, the survey information comprising quality of life survey answers for questions associated with a quality of life category;
automatically processing the survey information using an Artificial Intelligence (AI) alert decision support system to perform operations comprising:
generating an embedding for the survey information; and
using a model corresponding to the quality of life category to predict a change in a quality of life score for the patient for the quality of life category based on the embedded survey information;
comparing the change in the quality of life score for the patient with a threshold; and
generating an alert based on the comparison of the change in the quality of life score for the patient with the threshold.

18. The computer program product of claim 17, wherein the quality of life category is a health category, an emotion category, or an activity category.

19. The computer program product of claim 17, wherein the operations further comprise:
collecting the survey information in concert with distributing a drug product to a patient via an Internet of Things (IoT) device.

20. The computer program product of claim 19, wherein collecting the survey information comprises:
receiving the answers to the quality of life survey questions from the patient via the IoT device.
